(19) United States
(12) Patent Application Publication
(10) Pub. No.: US 2025/0195633 A1
(43) Pub. Date: Jun. 19, 2025

(54) ANTIGEN-SPECIFIC THERAPY FOR AUTOIMMUNE DISEASES

(71) Applicant: BODHI BIO LLC, Middletown, DE (US)

(72) Inventor: Animesh Sinha, Amherst, NY (US)

(73) Assignee: BODHI BIO LLC, Middletown, DE (US)

(21) Appl. No.: 18/849,006

(22) PCT Filed: Mar. 23, 2023
(86) PCT No.: PCT/US2023/016055

§ 371 Date: Sep. 20, 2024

Related U.S. Application Data
(60) Provisional application No. 63/322,702, filed on Mar. 23, 2022.
(60) Provisional application No. 63/322,699, filed on Mar. 23, 2022.

(51) Int. Cl. A61K 39/00; A61K 31/704; A61K 47/64; C07K 14/47; C07K 14/78; C12N 9/12

(52) U.S. Cl. CPC A61K 39/0008; A61K 31/704; A61K 47/6425; C07K 14/4713; C07K 14/78; C12N 9/12; A61K 2039/577; C12Y 207/10001

(57) ABSTRACT

A platform for therapy provides a therapeutic molecule comprising an auto-antigen linked by a linker to a toxin or other ablative molecule. Exemplary auto-antigen is Dsg3 or Dsg1. Other auto-antigens include Dsg1, Dsg3, bullous pemphigoid antigen 180, bullous pemphigoid antigen 230, MuSK, anti-phospholipase A2 Receptor, Factor VIII, one or more protein expressed by a viral vector administered to a subject for gene therapy, a DNA molecule carried by a gene therapy viral vector. A pharmaceutical composition comprising a disclosed therapeutic molecule is disclosed. Methods of treating diseases using the disclosed molecules are disclosed.

### TECHNICAL FIELD

This disclosure relates to a platform for using a therapeutic molecule as therapy for autoimmune diseases.

### BACKGROUND

Autoimmune disease is prevalent in the population and thus a major healthcare burden. Autoimmune disease is characterized by the host's immune system attacking one or more self-antigens. Treatment options for autoimmune diseases are limited and largely non-specific.

### SUMMARY

In one aspect, this disclosure provides a therapeutic molecule comprising an auto-antigen linked by a linker to a toxin or other ablative molecule. In some embodiments, the the auto-antigen is Dsg3 or Dsg1, or both. In other embodiments, the auto-antigen is bullous pemphigoid antigen 180, bullous pemphigoid antigen 230, or both. Yet in other embodiments, the auto-antigen is muscle specific tyrosine kinase (MuSK). Yet in other embodiments, the auto-antigen is an anti-phospholipase A2 Receptor. The auto-antigen can be any auto-antigen for an autoimmune disease where the auto-antigen is known.

In another aspect, this disclosure provides a pharmaceutical composition comprising a disclosed therapeutic molecule.

In another aspect, this disclosure provides a method of treating an autoimmune disease in a patient comprising administering a pharmaceutical composition comprising a therapeutically effective amount of a disclosed therapeutic molecule to said patient. In some embodiments, the method is one of treating Pemphigus vulgaris (PV) or its variants in a patient in need thereof, comprising administering a pharmaceutical composition comprising a therapeutically effective amount of a therapeutic molecule of a toxin or other ablative molecule linked by a linker to Dsg3 or Dsg1, or both, to said patient. In other embodiments, the method is a method of treating bullous pemphigoid in a patient in need thereof, comprising administering a pharmaceutical composition comprising a therapeutically effective amount of a therapeutic molecule of a toxin or other ablative molecule linked by a linker to bullous pemphigoid antigen 180, bullous pemphigoid antigen 230, or both, to said patient. In another embodiments, the method is a method of treating a subtype of myasthenia gravis in a patient in need thereof, comprising administering a pharmaceutical composition comprising a therapeutically effective amount of a therapeutic molecule of a toxin or other ablative molecule linked by a linker to muscle specific tyrosine kinase (MuSK) to said patient. In another embodiment, the method is a method of treating membranous nephropathy in a patient in need thereof, comprising administering a pharmaceutical composition comprising a a therapeutically effective amount of a therapeutic molecule of of a toxin or other ablative molecule linked by a linker to an anti-phospholipase A2 Receptor to said patient.

This disclosure also provides a therapeutic molecule comprising Factor VIII linked by a linker to an ablative molecule or a toxin. A pharmaceutical composition comprising a therapeutically effective amount of said therapeutic molecule is also provided.

This disclosure also provides a method to restore Factor VIII therapy to a patient in need thereof comprising administering a therapeutically effective amount of a pharmaceutical composition of an ablative molecule or a toxin to Factor VIII to said patient.

This disclosure also provides a therapeutic molecule comprising a protein expressed by a viral vector administered to a subject for gene therapy or to a DNA molecule carried by a viral vector administered to a subject for gene therapy linked by a linker to an ablative molecule or a toxin. A pharmaceutical composition comprising a therapeutically effective amount of said therapeutic molecule is also provided.

This disclosure also provides a method to improve gene therapy in a patient in need thereof comprising administering a therapeutically effective amount of a pharmaceutical composition of claim 20 to said patient.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1 is a schematic drawing of an embodiment of the disclosed therapeutic molecule and method of its use.

FIG. 2A (patient PV327), FIG. 2B (patient PV102), and FIG. 2C (patient PV114) show IgG reactivity in a longitudinal analysis for 3 patients. The y axis shows fold change in expression levels of the various autoantibodies in the blood. The X-axis shows data point for same patient in different phases of disease: A=active disease; LTR=long term remission (>6m).

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. “Effective amount,” “prophylactically effective amount,” or “therapeutically effective amount” refers to an amount of an agent or composition that provides the desired biological, therapeutic, and/or prophylactic result. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations.

As used herein, a “patient” and a “subject” are interchangeable terms and may refer to a human patient/subject, a dog, a cat, a non-human primate, etc.

The term “engineered protein” is known in the art. Briefly, the term “engineered protein” can refer to a polypeptide that is not naturally encoded by an endogenous nucleic acid present within an organism (e.g., a mammal). Examples of engineered proteins include modified enzymes with one or more amino acid substitutions, deletions, insertions, or additions that result in an increase in stability and/or catalytic activity of the engineered enzyme, fusion proteins, humanized antibodies, chimeric antibodies, divalent antibodies, trivalent antibodies, four binding domain antibodies, a diabody, and antigen-binding proteins that contain at least one recombinant scaffolding sequence.

The terms “polypeptide,” “peptide,” and “protein” are used interchangeably and are known in the art and can mean any peptide-linked chain of amino acids, regardless of length or post-translational modification.

The terms “self-antigen” and “auto-antigen” are used interchangeably herein.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Autoimmune Diseases

Autoimmune disease, prevalent in the population, is a major healthcare burden. An autoimmune disease is when a host's immune system attacks one or more self-antigens (auto-antigens). In the majority of autoimmune diseases the target auto-antigen(s) are not known. There are over 100 known human autoimmune diseases, affecting between 5-10% of the population. Autoimmune diseases are the 2^{nd} or 3^{rd} leading cause of morbidity and mortality and cost the US healthcare system over $100 billion annually. Lupus, pemphigoid, myasthenia gravis, multiple sclerosis, type 1 diabetes, and pemphigus vulgaris are just some examples of autoimmune diseases. Treatment options for autoimmune diseases are limited and largely non-specific or symptom oriented. No true cures are available and there is no consensus treatment guidelines. Targeted, individualized therapies are lacking.

Pemphigus is a group of IgG-mediated autoimmune diseases of stratified squamous epithelia, such as the skin and oral mucosa, in which acantholysis (the loss of cell adhesion) causes blisters and erosions. Pemphigus has three major subtypes: pemphigus vulgaris, pemphigus foliaceus and paraneoplastic pemphigus.

Pemphigus vulgaris (PV) is a potentially life-threatening autoimmune blistering skin disease, characterized by intraepithelial (suprabasalar) acantholysis, which is a loss of cell-cell adhesion. Quite a bit is known about PV. HLA genetic predisposition (HLA DRB1*0402 and DQB1*0503) is known; T cell (Th₂ driven) and B cell subsets (producing IgG4 autoantibodies) are known. And the primary autoantibody targets (autoantigens)—Desmoglein (Dsg)-3 and Desmoglein-1—are known.

Dsg3 and Dsg1 are keratinocyte-associated cell surface proteins relevant to cell-cell adhesion. Anti-Dsg3 and anti-Dsg1 autoantibodies can be detected in human PV patients and can be followed by ELISA. The titers roughly correlate with disease activity and serves as disease biomarkers.

Current and proposed treatments of PV include general immunosuppression with, for example, steroids; immunoglobulin-focused therapy, such as intravenous IG or FcRn blockade; B-cell targeted therapies, such as anti-CD20 molecules, BTK inhibitors, or BAFF inhibitors; and antigen-specific therapies.

Currently proposed antigen-specific therapy makes use of Chimeric Auto-Antibody Receptor T (CAAR-T) cells, an adaptation of the CAR-T cell strategy. CAAR-T cells are T-cells engineered to express autoantigen-based chimeric immunoreceptors. This platform directs T cells to kill autoreactive B lymphocytes through the specificity of the B cell receptor (BCR), without the requirement for T-cells (autologous or allogeneic). For PV, engineered human T cells expressing the PV autoantigen Dsg3 exhibit specific cytotoxicity against cells expressing anti-Dsg3 BCRs in vitro and specifically eliminate Dsg3-specific B cells in vivo in a PV mouse model.

Major hurdles, however, are associated with such cell-based therapies. These hurdles include:
complicated manufacturing process, involving harvesting of autologous T cells, engineering of cells, reinfusing cells and failing productions;
treatment time lag from start to finish;
complex patient referral pathway;
accredited CAAR T cell specialty centers and trained staff are needed;
potential for significant, life-threatening adverse effects;
potential for long lived, permanence of therapy;
inability to tune down therapy;
inability to readily adapt to multiple target therapy;
inability to readily personalize to individual patients;
inability to readily adapt to evolving autoimmune response in a given patient;
exorbitant costs;
commercial scalability challenges; and
complicated payer policies.

### Hemophilia

Hemophilia A patients can be treated successfully with factor VIII. However, 5-30% of patients with hemophilia A (of all severities) develop inhibitory anti-factor VIII antibodies (inhibitors) following replacement therapy.

### Gene Therapy Vectors

Gene therapy holds enormous promise. However, a patient's immune system can be a hindrance to gene therapy. Viral capsids, viral-vector DNA (also referred herein as a DNA molecule carried by a viral vector), and even the transgene products themselves may be recognized as foreign by the immune system. Immunity against viral capsids, viral-vector DNA (also referred herein as a DNA molecule carried by a viral vector), and transgene products can limit the efficacy and restrict dosing of gene therapy.

### Molecules, Compositions and Methods

In one aspect, this disclosure provides a therapeutic molecule comprising an auto-antigen linked by a linker to an ablative molecule or a toxin. In some embodiments, the the auto-antigen is Dsg3 or Dsg1, or both. In other embodiments, the auto-antigen is bullous pemphigoid antigen 180, bullous pemphigoid antigen 230, or both. Yet in other embodiments, the auto-antigen is muscle specific tyrosine kinase (MuSK). Yet in other embodiments, the auto-antigen is an anti-phospholipase A2 Receptor. The auto-antigen can be any auto-antigen for an autoimmune disease where the auto-antigen is known.

In another aspect, this disclosure provides a pharmaceutical composition comprising a disclosed therapeutic molecule.

In another aspect, this disclosure provides a method of treating an autoimmune disease in a patient comprising administering a pharmaceutical composition comprising a therapeutically effective amount of a disclosed therapeutic molecule to said patient. In some embodiments, the method is one of treating Pemphigus vulgaris (PV) or its variants in a patient in need thereof, comprising administering a pharmaceutical composition comprising a therapeutically effective amount of a therapeutic molecule of a toxin or other ablative molecule linked by a linker to Dsg3 or Dsg1, or both, to said patient. In other embodiments, the method is a method of treating bullous pemphigoid in a patient in need thereof, comprising administering a pharmaceutical composition comprising a therapeutically effective amount of a therapeutic molecule of a toxin or other ablative molecule linked by a linker to bullous pemphigoid antigen 180, bullous pemphigoid antigen 230, or both, to said patient. In another embodiments, the method is a method of treating a subtype of myasthenia gravis in a patient in need thereof, comprising administering a pharmaceutical composition comprising a therapeutically effective amount of a therapeutic molecule of a toxin or other ablative molecule linked by a linker to muscle specific tyrosine kinase (MuSK) to said patient. In another embodiment, the method is a method of treating membranous nephropathy in a patient in need thereof, comprising administering a pharmaceutical composition comprising a a therapeutically effective amount of a therapeutic molecule of of a toxin or other ablative molecule linked by a linker to an anti-phospholipase A2 Receptor to said patient.

This disclosure also provides a therapeutic molecule comprising Factor VIII linked by a linker to an ablative molecule or a toxin. A pharmaceutical composition comprising a therapeutically effective amount of said therapeutic molecule is also provided.

This disclosure also provides a method to restore Factor VIII therapy to a patient in need thereof comprising administering a therapeutically effective amount of a pharmaceutical composition of an ablative molecule or a toxin linked by a linker to Factor VIII to said patient.

This disclosure also provides a therapeutic molecule comprising a protein expressed by a viral vector administered to a subject for gene therapy or to a DNA molecule carried by a viral vector administered to a subject for gene therapy linked by a linker to an ablative molecule or a toxin. A pharmaceutical composition comprising a therapeutically effective amount of said therapeutic molecule is also provided.

This disclosure also provides a method to improve gene therapy in a patient in need thereof comprising administering a therapeutically effective amount of a disclosed pharmaceutical composition to said patient. Given that gene therapy often is given only once, in certain embodiments, this method is used at about the same time, or shortly before or shortly after, as the gene therapy itself. In some embodiments, the disclosed method improves gene therapy efficacy and allows normal dosing of gene therapy.

The viral vector for gene therapy includes, without limitation, adenovirus vector, adeno-associated vector, retroviral vector, herpes simplex viral vector, etc. The protein expressed by the viral vector includes a viral capsid protein. The protein expressed by the viral vector includes fragments thereof, mutants thereof or variants thereof, but those that still bind to the auto-antibodies. The viral DNA (a DNA molecule carried by a viral vector) can be the entire viral genome or mutants/variants thereof, a part of the viral genome or mutants/variants thereof, but those that still bind to the auto-antibodies.

In further embodiments, epitope mapping of the various proteins of the vector, such as an adeno-associated vector (AAV) reveals frequently targeted epitope or region on the viral protein that elicits an auto-immune response. That epitope or region can then be used as part of the disclosed multi-target therapeutic molecule.

In some embodiments, one or more linkers link one or more ablative molecules and/or toxins to one more more auto-antigens.

In some embodiments, the disclosed therapeutic molecule has an antibody backbone.

Any suitable toxin or other ablative molecule is contemplated. In some embodiments, the toxin is daunomycin or another anthracycline. Other toxins include, with limitation, pseudomonas exotoxin, diphtheria toxin and ribosome-inactivating proteins, a bleomycin. In some embodiments, the ablative molecule is a nano-molecule embedded with a toxin. The toxin is any suitable toxin and the ablative molecule is any suitable ablative molecule. The toxin or ablative molecule is one that can be linked by a linker to an auto-antigen. These are compounds known in the art as toxin and ablative molecule and can be obtained or made by routine methods.

Any suitable linker can be used. In some embodiments, the linker is acid sensitive. The linker is one that generally makes a covalent bond or covalent bonds with the protein(s). The linker is one that links two protein molecules or fragments together.

In some embodiments, the linker is a peptide. A peptide linker can be composed of small, non-polar (e.g., Gly) or polar (e.g., Ser or Thr) amino acids. The linker can be poly-glycine or poly-glycine with one or more Ser and/or Thr. A peptide linker can be generated as part of the multi-target therapeutic molecule by recombinant DNA technology.

In other embodiments, the linker is a chemical non-peptide moiety. Proteins are typically cross-linked in a chemical reaction involving a cross-linker and side chains of amino acids. The reactivity of amino groups, thiols and carboxylic acids, render them as prime targets for cross-linking. The cross-linker can be a molecule with two reactive groups on either end, separated by a spacer. These reactive groups can target either primary amino groups (found in the side chain of lysine and at the protein N-terminus) or thiols (cysteine side chain). A small molecule, 1-Ethyl-3-(3-dimethylaminopropyl) carbodiimide (EDC), can be used to activate carboxylic acids (aspartate, glutamate, protein C-terminus) to cross-link with amines (lysine, protein N-terminus). This directly cross-links atoms of the protein(s) with each other in a “zero-length” cross-link. Other cross-linkers have been synthesized by introducing N-hydroxyphthalimide, hydroxybenzotriazole, and 1-hydroxy-7-azabenzotriazole as leaving groups instead of the commonly used N-hydroxysuccimidyl moiety. Other examples of chemical linkers include, without limitation, Bis(sulfosuccinimidyl) suberate (BS3), polyethylene glycol (PEG), which can be used as a single or branched chained moiety in a pegylation reaction, block sulfhydryls, such as N-Ethylmaleimide (NEM) and S-methyl methanesulfonothioate (MMTS), N-Succinimidyl-S-acetylthioacetate (SATA), and 2-Iminothiolane-HCl (Traut's reagent). In some embodiments, the cross linker is an acid-sensitive cis-aconityl group, such as, for example, cis-aconitic anhydride. Diener, E., Diner, U., Sinha, A., Xie, S., and Vergidis, R. 1986, Science 231 (4734): 148-150; Diener, U., Diener, E., Sinha, A., Xie, S., and Vergidis, R. 1986. Selective suppression of murine lymphocyte function by daunomycin conjugated via an acid sensitive spacer to target specific carriers. In: Mediators of Immune Regulation and Immunotherapy (S. K. Singhal and T. L. Delovitch, Eds.), Elsevier Science Publications, Amsterdam, p. 177-181. These chemical non-peptide linkers are attached to proteins chemically by reactions known in the art. See, e.g., Id.

In embodiments where the linker is an acid sensitive linker, the linker is cleaved in an acidic intracellular environment, such as in the lysosome, releasing the toxin to kill the B cell. In some embodiments, the toxin is daunomycin. Diener, E., Diner, U., Sinha, A., Xie, S., and Vergidis, R. 1986. Science 231 (4734): 148-150; Diener, U., Diener, E., Sinha, A., Xie, S., and Vergidis, R. 1986. Selective suppression of murine lymphocyte function by daunomycin conjugated via an acid sensitive spacer to target specific carriers. In: Mediators of Immune Regulation and Immunotherapy (S. K. Singhal and T. L. Delovitch, Eds.), Elsevier Science Publications, Amsterdam, p. 177-181.

The auto-antigen can be an antigenic determinant of the auto-antigen, one that maintains the ability to bind to its B cell receptor. The auto-antigen, or its antigenic determinant, can be part of a fusion protein.

The auto-antigen(s) can be chimeric/fusion proteins. In some embodiments, the auto-antigen is fused to an Fc domain of an immunoglobulin. In some embodiments, the auto-antigen comprises N-terminal auto-antigen and C-terminal human IgG1 Fc domain.

In some embodiments, the auto-antigen(s) on the disclosed therapeutic molecule binds to B-cells expressing on their surface B-cell receptors to the auto-antigens, such as to Dsg3 and to Dsg1. In some embodiments, The auto-antigen on the disclosed therapeutic molecule serves as lure to attract auto-reactive B cells expressing auto-antigen specific B cell receptors on their surface.

The autoantigen can be a full-sized protein or smaller polypeptides (or variants/mutants of either a full-sized protein or smaller polypeptide) but still bind to its antibody or antigen-binding fragment thereof. In some embodiments, the size of the auto-antigen is optimized for binding to its cognate B-cell receptor but does not get bound by a patient's circulating auto-antibodies to the auto-antigen. The known auto-antigens are known in the art and can be obtained by, for example, recombinant DNA technology or other methods known in the art. The auto-antigens may be purchased or gifted.

Similar to bi-specific antibodies this approach facilitates antigen-specific elimination of autoreactive B-lymphocytes.

The disclosure provides a highly specific, potentially less toxic strategy to create a “targeted bullet” for the treatment of autoimmune diseases.

The disclosed molecule, method and system do not require harvesting of autologous patient lymphocytes; do not require genetic engineering of patient T cells; and do not require reinfusion of autologous T cells.

Autoimmune diseases with known auto-antigens include, without limitation, pemphigus, pemphigoid, myasthenia gravis (such as, for example and without limitation, acetylcholine receptors (AChRs), muscle-specific kinase (MuSK) and low-density lipoprotein receptor-related protein 4 (LRP4)), Type 1 Diabetes (such as, for example and without limitation, insulin, glutamic acid decarboxylase, and protein tyrosine phosphatase), and multiple sclerosis (myelin).

Methods of making the disclosed therapeutic molecules are known in the art. For example, recombinant DNA technology can be used to make the separate protein molecules; chemical reactions to link proteins to each other with a linker or a protein and a DNA together with a linker are known and are used to link these moieties to each other. In case of a peptide linker, the therapeutic molecule, or at least part of it, can be made as a large fusion protein.

### Formulating and Administering Compositions

The disclosed composition may be administered to a subject in need thereof by any suitable mode of administration, any suitable frequency, and at any suitable, effective dosage.

The composition for use in a disclosed method may be in any suitable form and may be formulated for any suitable means of delivery.

In some embodiments, the disclosed composition is provided in a form suitable for injection, such as subcutaneous, intramuscular, intravenous, intraperitoneal, or any other route of injection. In some embodiments, compositions for injection are provided in sterile and/or non-pyrogenic form and may contain preservatives and/or other suitable excipients, such as sucrose, sodium phosphate dibasic heptahydrate or other suitable buffer, a pH-adjusting agent such as hydrochloric acid or sodium hydroxide, and polysorbate 80 or other suitable detergent.

When provided in solution form, in some embodiments, the composition for use in a disclosed method is provided in a glass or plastic bottle, vial or ampoule, any of which may be suitable for either single or multiple use. The bottle, vial or ampoule containing the disclosed composition may be provided in kit form together with one or more needles of suitable gauge and/or one or more syringes, all of which preferably are sterile. Thus, in certain embodiments, a kit is provided comprising a liquid solution as described above, which is packaged in a suitable glass or plastic bottle, vial or ampoule and may further comprising one or more needles and/or one or more syringes. The kit may further comprise instruction for use.

The composition for use in a disclosed method can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the composition for use in a disclosed method comprise at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N. J. 1991) or other standard pharmaceutical science texts, such as the Handbook of Pharmaceutical Excipients (Shesky et al. eds., 8th ed. 2017).

In some embodiments, the composition for use in a disclosed method can be administered intra-gastrically, intravenously, intraperitoneally or intramuscularly, but other routes of administration are also possible.

Water may be used as a carrier and diluent in the composition. The use of other pharmaceutically acceptable solvents and diluents in addition to or instead of water is also acceptable.

Large macromolecules that are slowly metabolized, such as proteins, polysaccharides, polylactic acids, polyglycolic acids, polymeric amino acids, copolymers of amino acids, can also be used as carrier compounds for the composition. Pharmaceutically acceptable carriers in therapeutic compositions may additionally contain liquids, such as water, saline, glycerol or ethanol. Moreover, the said compositions may further comprise excipients, such as wetting agents or emulsifiers, buffering substances, and the like. Such excipients include, among others, diluents and carriers conventional in the art, and/or substances that promote penetration of the active compound into the cell, for example, DMSO, as well as preservatives and stabilizers.

The composition for use in a disclosed method may be presented in various dosage forms depending on the object of application; in particular, it may be formulated as a solution for injections.

The composition for use in a disclosed method may be administered systemically. Suitable routes of administration include, for example, or parenteral administration, such as intravenous, intraperitoneal, intragastric as well as via drinking water. However, depending on a dosage form, the disclosed composition may be administered by other routes.

The disclosed composition can be co-administered with another appropriate agent or therapy.

### EXAMPLES

For this invention to be better understood, the following examples are set forth. These examples are for purposes of illustration only and are not to be construed as limiting the scope of the invention in any manner.

### Example 1 Assessment of Auto-Ab Specificity in PV Patients—Development of a Multiplexed Platform to Comprehensively Identify Autoantigens in an Autoimmune Disease

Multiplexed protein microarrays were used to probe PV patient or negative control sera.

Array 1.0:15 auto-antigens were tested on 80 patients/controls; 5 disease associated targets were identified.
Sajda T., Hazelton J., Patel M., Seiffert-Sinha K. Steinman L., Robinson W. H., Haab B. B., and Sinha A. A. 2016. Multiplexed autoantigen microarrays identify HLA as a key driver of anti-desmoglein and-non-desmoglein reactivities in Pemphigus. PNAS 113 (7): 1859-64. http://www.pnas.org/content/113/7/1859.long
Sinha, A. A. and Sajda, T. 2018. The evolving story of autoantibodies in Pemphigus vulgaris: development of the “super compensation hypothesis”. Front. Med. 5:218. doi: 10.3389/fmed.2018.00218 [Epub ahead of print].

Array 2.0:50 auto-antigens were tested on 675 patients/controls; 35 disease-associated targets were identified.

TABLE 1

| | Antigens | Samples Screened |
|---|---|---|
| Array 1.0 | 15 | 80 (40 patient, 40 controls) |
| Array 2.0 | 50 | 675 (459 patient, 216 control) |

Reactivities were stratified by clinical subtypes, with static parameters such as age, sex, HLA expression and disease onset, and with dynamic parameters such as disease activity, morphology, and disease duration.

See Sajda, T et al. Proc Natl Acad Sci. 2016 Feb. 16; 113 (7): 1859-64.

IgG Reactivity was compared for PV patients vs. controls. Thirty five antigens were identified with significantly increased IgG autoreactivity in the PV group. These auto-antigens are shown in Table 2.

TABLE 2
AUTO-ANTIGENS

| | CHRNE | | | | | |
|---|---|---|---|---|---|---|
| ATP2c1 | (Cholinergic | | | | | |
| (ATPase | Receptor | | | CHRM3 | | |
| Secretory | Nicotinic | Dsg1 | | (cholinergic | | DSC3 |
| Pathway Ca2+ | Epsilon | (Desmoglein | | receptor | ANXA9 | (Desmocollin- |
| Transporting 1) | Subunit) | 1) | HLA E | muscarinic 3) | (Annexin A9) | 3) |
| CD33 | Chromatin | Dsg3 | HLA-DRA | CHRM4 | C5a1 | PG |
| | | (Desmoglein | | (cholinergic | | |
| | | 3) | | receptor | | |
| | | | | muscarinic 4) | | |
| CENP A/B | COL3A1 | FcIgE | HSP60 | CHRM5 | COL21A1 | SOD2 |
| | | | | (cholinergic | (collagen type | (superoxide |
| | | | | receptor | XXI alpha 1 | dismutase 2) |
| | | | | muscarinic 5) | chain) | |
| CHRNA9 | Collagens | FH (fumarase) | CHRM1 | NDUFS1 | DSC1 | TG |
| | | | (cholinergic | (NADH- | (Desmocollin- | (thyroglobulin) |
| | | | receptor | ubiquinone | 1) | |
| | | | muscarinic | oxidoreductase) | | |
| | | | 1) | | | |
| CHRND | CYB5B | GP1BA | CHRM2 | PDHA1 | DSC2 (Desmocollin- | Peroxidase) |
| (Acetylcholine | (Cytochrome | (Glycoprotein | (cholinergic | (pyruvate | 2) | TPO (Thyroid |
| receptor | B5 Type B) | Ib Platelet | receptor | dehydrogenase | | |
| subunit delta) | | Subunit | muscarinic | E1 subunit | | |
| | | Alpha) | 2 | alpha 1) | | |

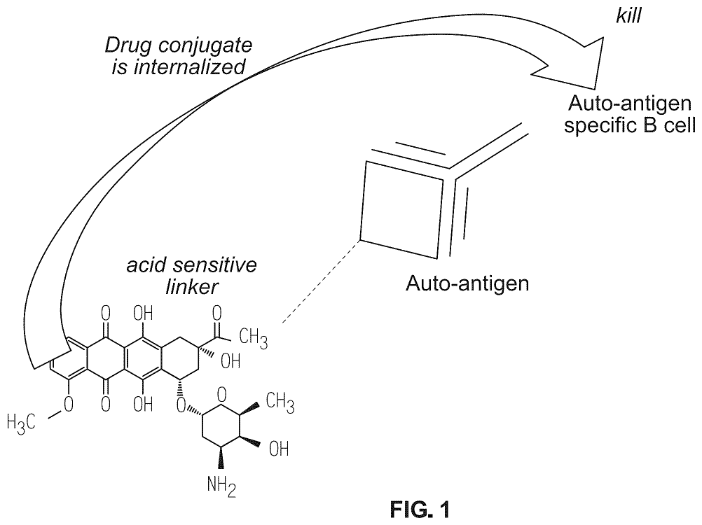

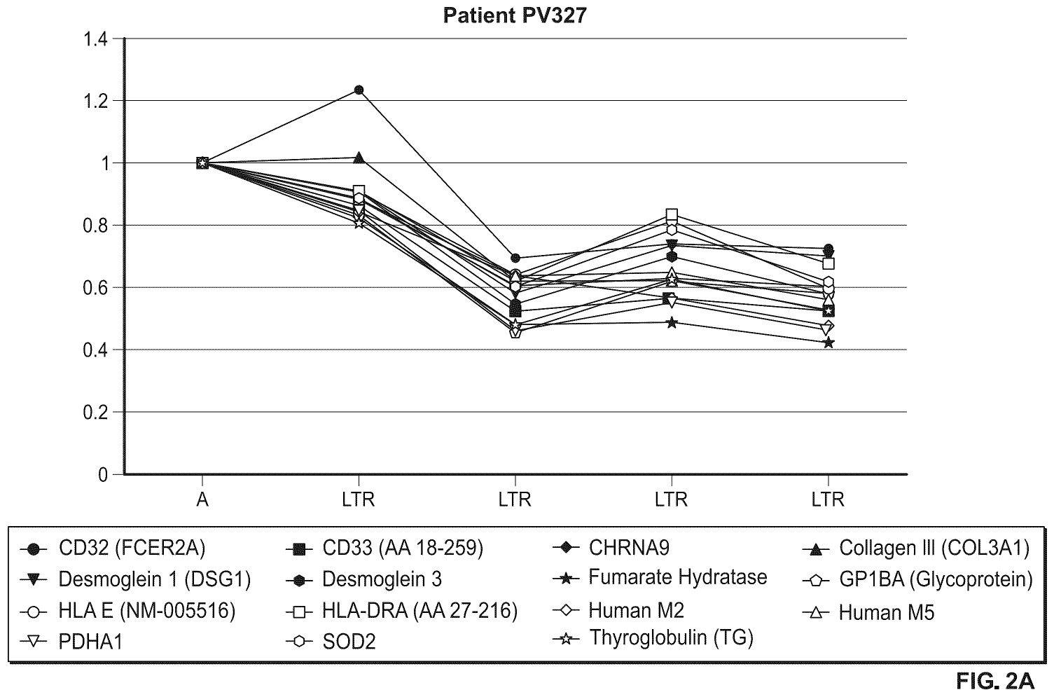

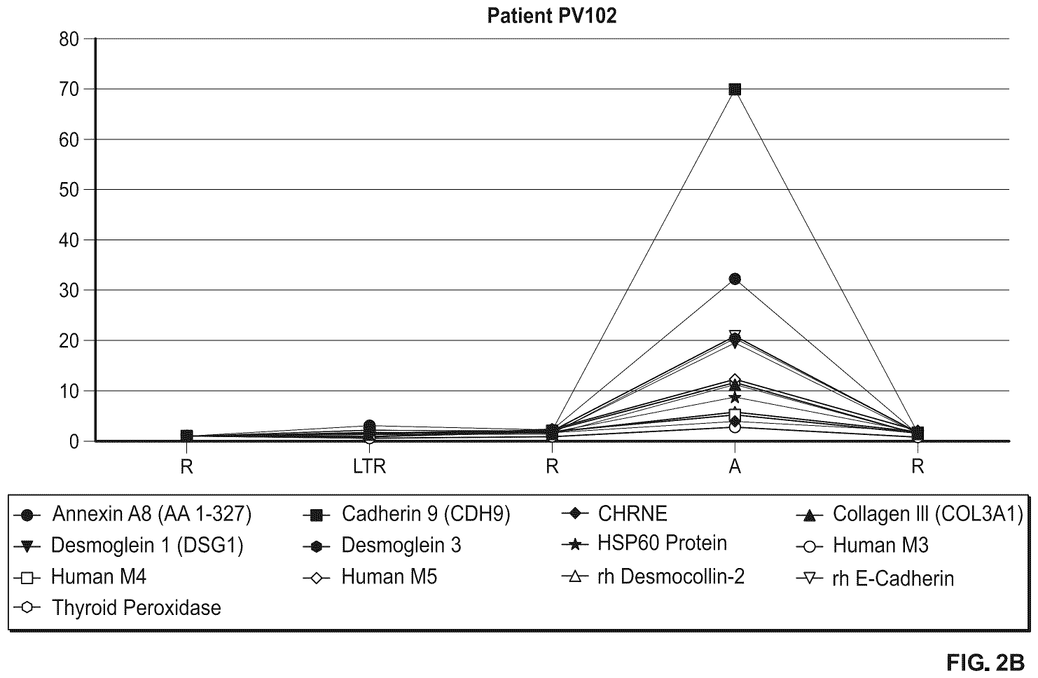

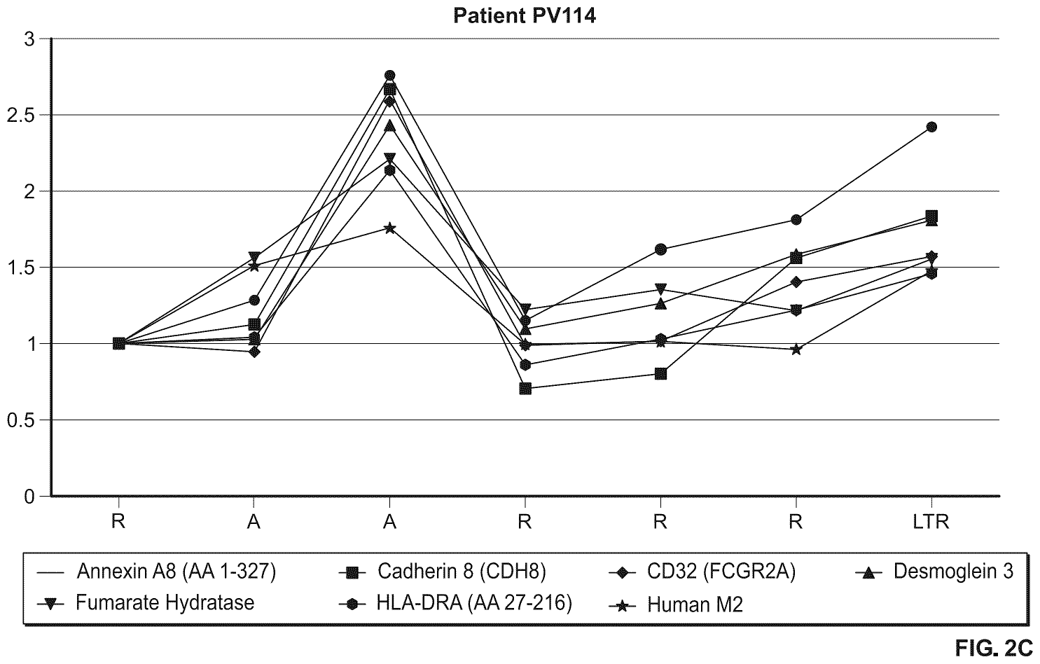

Multiple non Dsg3 and Dsg1 auto-antibodies were found to be correlated with disease activity. The pattern is similar in each patient, with an average of 9 auto-antigens. It appears that the set of antigens driving disease activity differs in each patient. Individual patients have unique auto-antigenic profiles. See FIGS. 1A, 1B, and 1C.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A therapeutic molecule comprising an auto-antigen linked by a linker to an ablative molecule or a toxin.

2. The molecule of claim 1, wherein the auto-antigen is Dsg3 or Dsg1.

3. The molecule of claim 1, wherein the auto-antigen is bullous pemphigoid antigen 180, bullous pemphigoid antigen 230, or both.

4. The molecule of claim 1, wherein the auto-antigen is muscle specific tyrosine kinase (MuSK).

5. The molecule of claim 1, wherein the auto-antigen is an anti-phospholipase A2 Receptor.

6. The molecule of claim 1, wherein the linker is acid sensitive.

7. The molecule of claim 1, wherein the toxin is daunomycin.

8. A pharmaceutical composition comprising a therapeutic molecule of claim 1.

9. A method of treating an autoimmune disease in a patient comprising administering a pharmaceutical composition comprising a therapeutically effective amount of a therapeutic molecule of claim 1 to said patient.

10. A method of treating Pemphigus vulgaris (PV) or its variants in a patient in need thereof, comprising administering a pharmaceutical composition comprising a therapeutically effective amount of a therapeutic molecule of claim 2 to said patient.

11. A method of treating bullous pemphigoid in a patient in need thereof, comprising administering a pharmaceutical composition comprising a therapeutically effective amount of a therapeutic molecule of claim 3 to said patient.

12. A method of treating a subtype of myasthenia gravis in a patient in need thereof, comprising administering a pharmaceutical composition comprising a therapeutically effective amount of a therapeutic molecule of claim 4 to said patient.

13. A method of treating membranous nephropathy in a patient in need thereof, comprising administering a pharmaceutical composition comprising a a therapeutically effective amount of a multi-target therapeutic molecule of claim 5 to said patient.

14-21. (canceled)
